(19) United States
(12) Patent Application Publication
(10) Pub. No.: US 2004/0022880 A1
(43) Pub. Date: Feb. 5, 2004

(54) Extracts of mixed arctium lappa L., carrot and whole radish for treating hypertension, constipation and detoxification

(76) Inventor: Yuan Shi, Little Neck, NY (US)

Correspondence Address: Albert Wai-Kit Chan, Law Ofices of Albert Wai-Kit Chan, LLC, World Plaza, Suite 604, 141-07 20th Avenue, Whitestone, NY, 11357, US

(21) Appl. No.: 10/213,468

(22) Filed: Aug. 5, 2002

(51) Int. Cl. A61K 35/78

(52) U.S. Cl. 424/764; 424/773

(57) ABSTRACT

A mixed substance for treating hypertension and constipation, for detoxification, and for improving the immune system, produced by extracting arctium lappa L., carrot, and whole radish with water one or two hours at temperatures of 70° C.-100° C. under agitation, separating the extracts and solid by-products, vacuum condensing the extracts, lyophilizing condensed extracts to a powder using a low temperature, and encapsulating the powder or pressing the powder into tablets. Patients taking a daily dosage of this vegetable medicine have shown greatly improved health conditions.

This application claims priority of U.S. Ser. No. 09/824,925, filed Apr. 3, 2001, the content of which is incorporated by reference here into this application.

Throughout this application, various references are referred to. Disclosure of these publications in their entireties are hereby incorporated by reference into this application to more fully describe the state of the art to which this invention pertains.

### FIELD OF THE INVENTION

This invention relates to a vegetable medicine designed specifically to cure sickness and improve health conditions.

### BACKGROUND OF THE INVENTION

This invention relates to extracts of arctium lappa L., carrot and whole radish (radish and radish leaves) having antihypertension, detoxification and constipation-treating properties.

Hypertension is the medical term for high blood pressure. It is defined in an adult as blood pressure greater than or equal to 140 mmHg systolic pressure or greater than or equal to 90 mmHg diastolic pressure. Blood pressure is measured in millimeters of mercury (mmHg). High blood pressure directly increases the risk of coronary heart disease (which leads to heart attack) and stroke, especially along with other risk factors.

High blood pressure can occur in children or adults, but it is more common among people over age 35. Medical science does not understand why most cases of high blood pressure occur, and it is difficult to say how to prevent it.

Current pharmaceutical treatments for essential hypertension include diuretics, beta-blockers, ACE inhibitors, angaotensin converting enzyme inhibitors, thiazid and calcium antagonists. Some of them were patented, such as U.S. Pat. No. 4,559,340. This patent is related to the manufacture of an anti-hypertension agent. The invention discloses benzothiadiazinyl and quinazolinyl substituted carboxylalkyl dipeptide, wherein the benzothiodiazinyl or quinazolinyl portions are joined to the dipeptide portions by an aminocarbonyl group. Compounds of this patent are used for the treatment of congestive heart failure and glaucoma. In addition, compounds of this patent also have diuretic activity.

U.S. Pat. No. 6,110,951: Thiazolidine derivatives for the treatment of hypertension. This patent is related to the second medical use of thiazolidine compounds having anti-diabetic properties. Thiazolidine derivatives are compounds which are not only used for the treatment of hypertension but also for the control of essential hypertension.

U.S. Pat. No. 4,929,641: Novel mercapto-acylamino acids useful in the treatment of hypertension and combinations of mercapto-acylamino acids and atrial natriuretic factors or angiotensin coverting enzyme inhibitors useful for treating hypertension are disclosed and mercapto-acylamino acids in the treatment of hypertension and combinations mercapto-acylamino acids and atrial natriuretic factors or angiotensin converting enzyme inhibitors for treating hypertension are described.

U.S. Pat. No. 4,513,009: Amino acid derivatives and their therapeutic applications. This invention is related to amino acid derivatives, and compositions containing the same having enkephalinase-inhibiting, antalgic, antidiarrhea and hypertension activities.

U.S. Pat. No. 6,096,759: This invention is related to the treatment of essential hypertension by administration of the anti-hypertensive effective of 4-hydroxy-2,2,6,6-tetramethyl-1-piperidine-1-oxyl (tempol).

Currently available anti-hypertensive agents are not without side effects, such as the elevation of blood lipids and glucose. The elevation of blood lipids and glucose by these agents has been suggested as a reason why anti-hypertensive agents have not demonstrated any benefits to patients being monitored in death rate studies.

Toxins Undermine Our Health defines a toxin simply as any substance that creates irritating and/or harmful effects in the body, undermining our health or stressing our biochemical or organ functions.

More specifically, a body overloaded with toxin can manifest a number of symptoms. These include constipation, stomach bloating, poor digestion, gas, fatigue, weight gain, excessive mucus, poor concentration, headache, poor skin, poor memory, depression, body odor, and bad breath.

Some health practitioners report toxins specific diseases. R. A. Buist, M. D., (International Clinical Nutrition; 1998; 8:4) states that chronic fatigue syndrome may be related to toxin exposure. Multiple chemical sensitivity and fibromyalgia (muscle and joint pain) may also be environment-related diseases.

Constipation is a symptom that refers to infrequent bowel movements, but it may also refer to a decrease in the volume or weight of stool, the need to strain to have a movement, a sense of incomplete evacuation, or the need for enemas, suppositories or laxatives in order to maintain regularity.

More serious causes of constipation include growths or areas of narrowing in the colon, so it is wise to seek the advice of a colon and rectal surgeon when constipation persists. Constipation may rarely be a symptom of scieroderma, lupus, or disorders of the nervous or endocrine systems, including thyroid disease, multiple sclerosis, Parkinson's Disease, stroke, and spinal cord injuries.

Many medications, including painkillers, antidepressants, tranquilizers, and other psychiatric medications, blood pressure medications, diuretics, iron supplements, calcium supplements, and aluminum containing antacides can cause or worsen constipation.

The vast majority of patients with constipation are treated by adding high-fiber foods like bran, shredded wheat, whole grain breads and certain fruits and vegetables to the diet, along with increased fluids.

In U.S. Pat. No. 5,948,824: A chemical agent of organic origin is used for reducing the changes in waistline or for reducing or eliminating constipation. U.S. Pat. No. 5,418,220: This invention uses dimethicone as an agent for treating constipation.

The invention disclosed herein relates to the use of mixed vegetables to treat hypertension, detoxify the body and treat constipation holistically. Its intent is to heal the entire body, instead of just treating the symptoms. The vegetables contain many natural compounds that play off one another, producing a wide variety of results. Even medical science does not always understand how the compounds work together, or even exactly what they all are.

This invention relates to the novel method of producing a mixed-vegetable medicine. The medicine includes arctium lappa L., carrot and whole radish. When extracted, arctium lappa L., carrot and whole radish have powerful functions, such as hypertension treatment, detoxification, and constipation relief.

### SUMMARY OF THE INVENTION

This invention provides a method of producing a medicinal substance for the treatment of hypertension, for detoxification, and for the treatment of constipation.

It has been discovered that arctium lappa L., carrot and whole radish extracts are active as anti-hypertension agents, anti-constipation agents and detoxification agents.

Carrot: Scientific Name: Garden carrot: Daucus carota Common Name: Wild carrot: daucus, Queen Anne' lace.

Carrots are rich in fiber and nutrient content. Contemporary herbalists add that it can also be used to promote healthy eyes, soothe indigestion, lower cholesterol, and help prevent cancer. In parts of the Europe today, people sip brews made from the root or dried above-ground parts of wild carrots to promote urination (as a mild diuretic, or “water”), prevent worms, and break up bladder stones. The seeds have been used in traditional medicine as a diuretic and to stimulate menstruation and relieve gas. In years past it was enlisted as a treatment for such wide-ranging ailments as worms, diabetes, and kidney disease, and was even considered an aphrodisiac.

Few sources dispute the nutritional richness of the edible carrot (and the root oil), which contains plentiful stores of beta-carotene, a plant form of vitamin A that the body relies on in many important ways. Many of the claims for carrot appear to be based on the presence of vitamin A, including its cancer-preventing potential. Most medicinal claims for carrots demand more research. Investigators are following several interesting leads, however. According to reports, carrot extracts dilate vessels and relax smooth muscles of animal organs isolated in the laboratory, slow heart activity in frog and dog hearts, and lower blood pressure by relaxing the smooth muscle in rats' hearts. The implications for treating liver, heart, or other diseases in humans still require extensive research. According to a 1995 study, carrot extract may merit investigation as a liver protectant. In mice, it helped shield the liver from carbon tetrachloride poisoning.

Evidence indicates that the carrot and carrot oils are safe to consume in moderation. Too much of either may be unwise, however. Carrot seeds contain a psychoactive agent called myristicin, which, if taken in large amounts, raises the risk for neurologic reactions. Skin irritation and blisters can develop after handling the leaves, especially if they are wet and you are then exposed to the sun.

Arctium lappa L. : Pharmaceutical name is fructus arctii lappae. Botanical name: Arctium lappa L. English mane is great burdock fruit, arctium. Pharmacological and clinical research have shown significant in vitro inhibitory effect against streptococcus pneumoniae and many pathogenic fungi. Endocrine effect: Ingredients taken from arctium lappa L. showed a significant and prolonged hypoglycemic effect in rats. Action and indication: Arctium lappa L. benefits the throat when there are symptoms such as fever, cough, and sore, red, swollen throat. It also clears heat and relieves toxicity.

Am J Chin Med 1996, 24(2): 127-37: Described and investigated the effects of arctium lappa L. on anti-inflammatory and free radical scavenger activity. Subcutaneous administration of A. lappa crude extract significantly decreased carrageenan-induced rat paw edema. When simultaneously treated with CC14, it produced pronounced activities against CC14-induced acute liver damage. The free radical scavenging activity of its crude extract was also examined by means of an electron spin resonance (ESR) spectrometer. The IC50 of A. lappa extract on superoxide and hydroxyl radical scavenger activity was 2.06 mg/ml and 11.8 mg/ml, respectively. These findings suggest that arctium lappa decreases carrageenan-induced paw edema and CC14-induced hepatotoxicity, possibly due to the scavenging effect of A. lappa.

Chem Pharm Bull (Tokyo) 1996 Dec; 44(12): 2300-4 reported the differentiation-inducing activities of lignoids from arctium lappa L. against mouse myeloid leukemia cells (M1).

Radish: Botanical Name: Raphanus sativus, family: N. O. Cruciferae.

### DETAILED DESCRIPTION OF THE INVENTION

### Methods

The amount of the mixed vegetable composition administered depends upon the percent of active ingredients within its formula, such as arctium lappa L., carrot and whole radish (radish and radish leaves).

Formula 1

A tablet composition for oral administration is prepared by combining the following ingredients:

| Ingredient | % W/W |
|---|---|
| Arctium lappa L. | 40 |
| Carrot | 60 |

Clean arctium lappa L. and carrot. Chop into small dices or any shape (use a chopping processor). Pour small dices into a batch container equipped with an agitator and heating jacket. Add purified water to the batch container. The amount of purified water is two times the amount of the total ingredients. Turn on agitator and heat to between 70° C. and 100° C. Extract one to two hours. After extracting, separate the liquid product and the solid by-product. Vacuum condense extracted liquid at a temperature not exceeding 100° C. Lyophilize condensed extracts to a powder using a low temperature. The final step is to encapsulate the powder or press the powder into tablets.

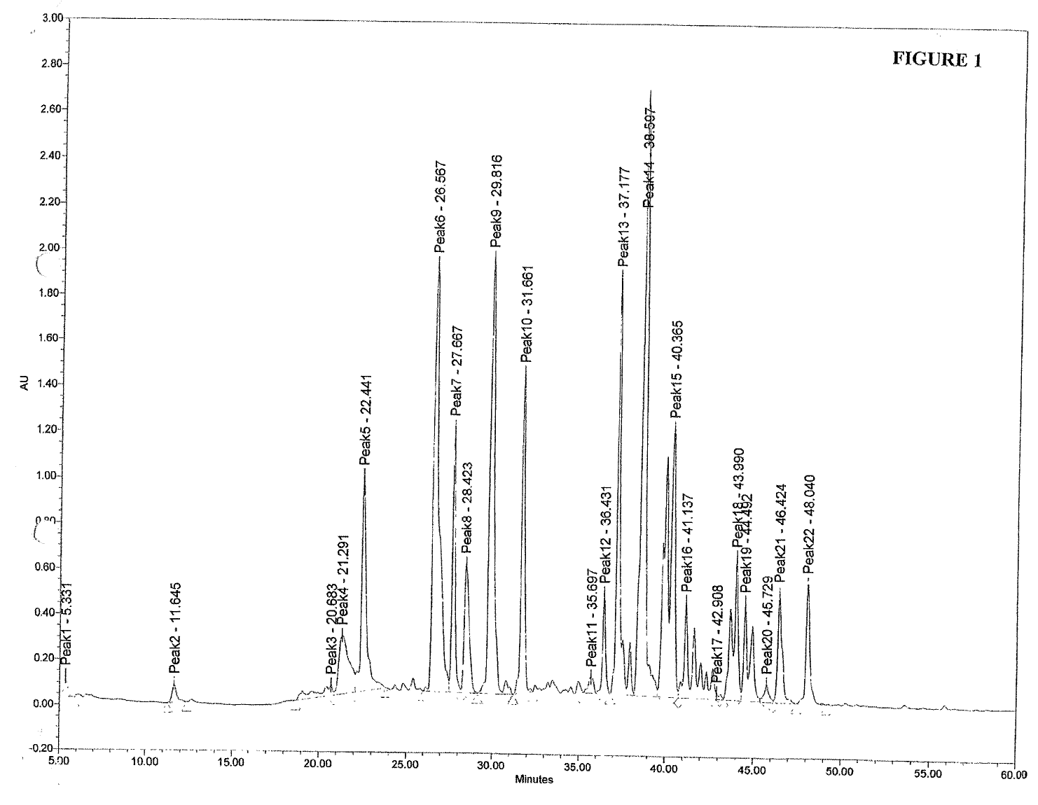

The HPLC (high performance liquid chromatography) fingerprints of FORMULA 1 extracts (FIG. 1).

C18 column, 300A, 5 uM, 4.6 mmID×250 mmL, Flow rate 1 mL/min, solvent A: 0.1% TFA/Water, solvent B: 0.1% TFA/Acetonitrile. 0-10 min 100% solvent A, 10-70 min 0%-50% solvent B, 70-100 min 50%-100% solvent B, 100-110 min 100%-0% solvent B.

Each tablet contains 500 mg. Administering one or two tablets, two times a day to a person in need of treatment provides an improved condition.

Formula 2

| Ingredient | % W/W |
|---|---|
| Arctium lappa L. | 20 |
| Carrot | 40 |
| Whole radish | 40 |

Clean arctium lappa L. ,carrot and whole radish. Chop into small dices or any shape (use a chopping processor). Pour small dices into a batch container equipped with an agitator and heating jacket. Add purified water to the batch container. The amount of purified water is two times the amount of the total ingredients. Turn on the agitator and heat to between 70° C. and 100° C. Extract one to two hours. After extracting, separate the liquid product and the solid by-product. Vacuum condense extracted liquid at a temperature not exceeding 100° C. Lyophilize condensed extracts to a powder using a low temperature. The final step is to encapsulate the powder or press powder into tablets.

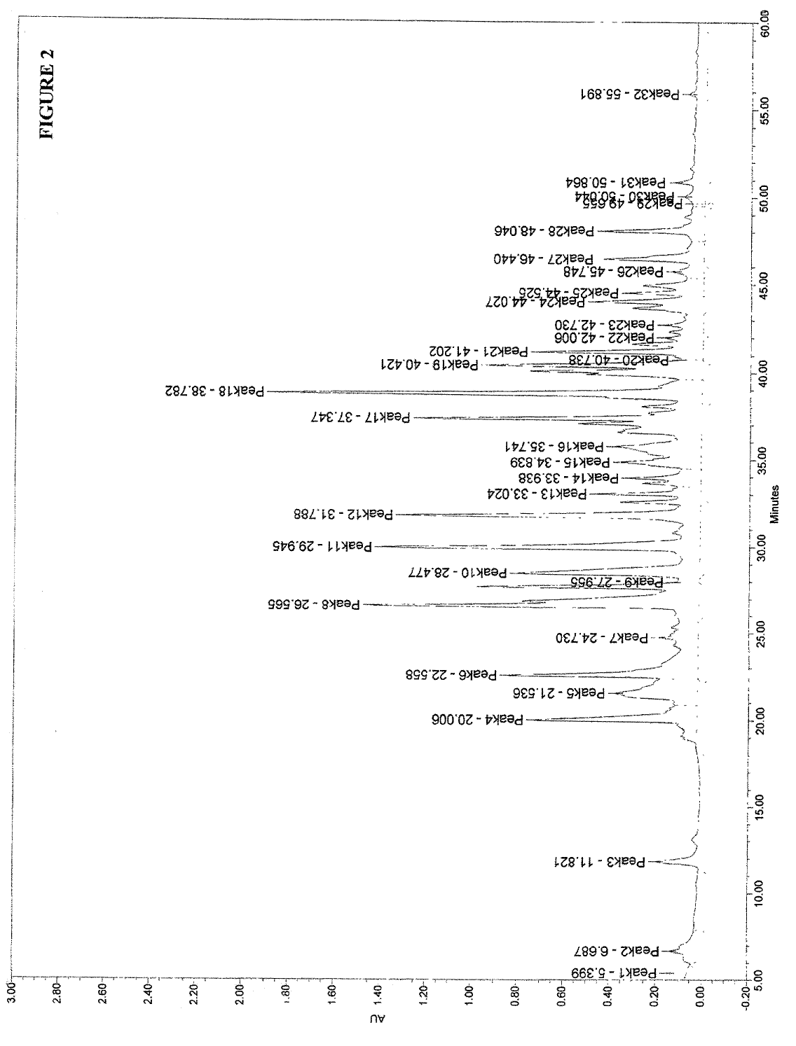

The HPLC (high performance liquid chromatography) fingerprints of FORMULA 2 extracts (FIG. 2).

C18 column, 300A, 5 uM, 4.6 mmID×250 mmL, Flow rate 1 mL/min, solvent A: 0.1% TFA/Water, solvent B: 0.1% TFA/Acetonitrile. 0-10 min 100% solvent A, 10-70 min 0%-50% solvent B, 70-100 min 50%-100% solvent B, 100-110 min 100%-0% solvent B.

FORMULA 2 extract toxicity studies, according to Procedures and Methods for Toxicological Assessment on Food Safety, showed no toxicity.

The conclusion is that the extracts of arctium lappa L., carrot and whole radish (radish and radish leaves), according to HPLC fingerprints like FIG. 1 and FIG. 2, have drug functions that are anti-hypertension, detoxifying and constipation-curing. Since the active ingredients derived from vegetables can vary from source to source, quality control of the individual components is required.

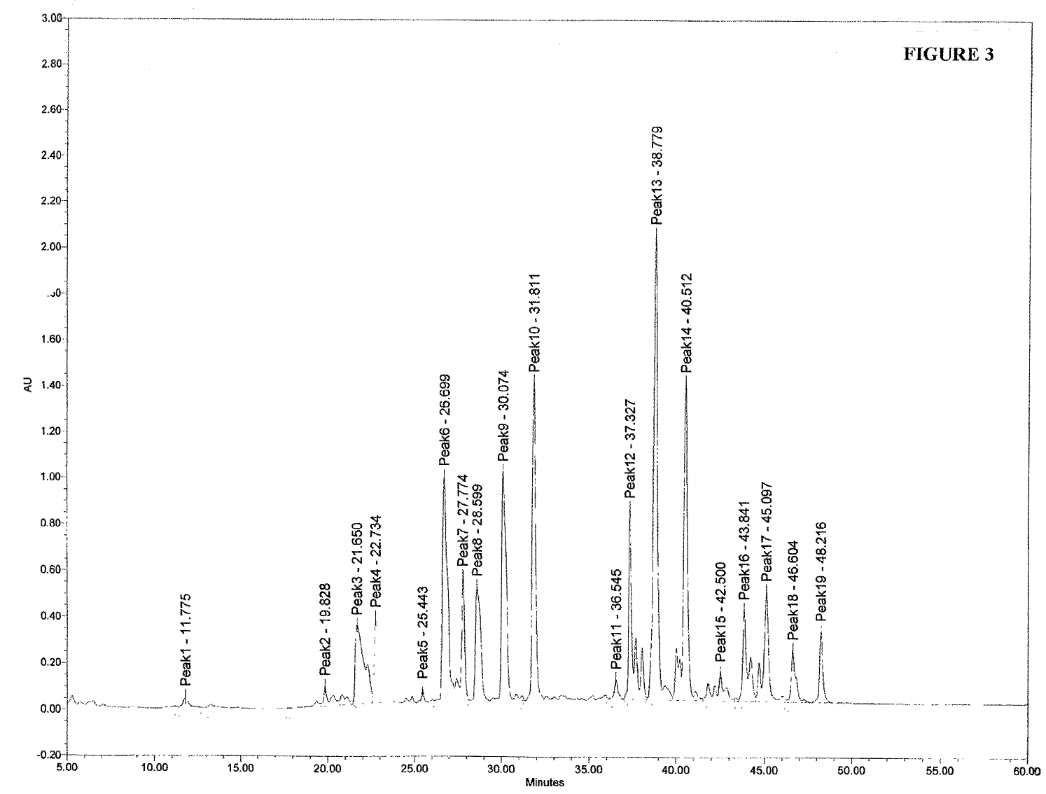

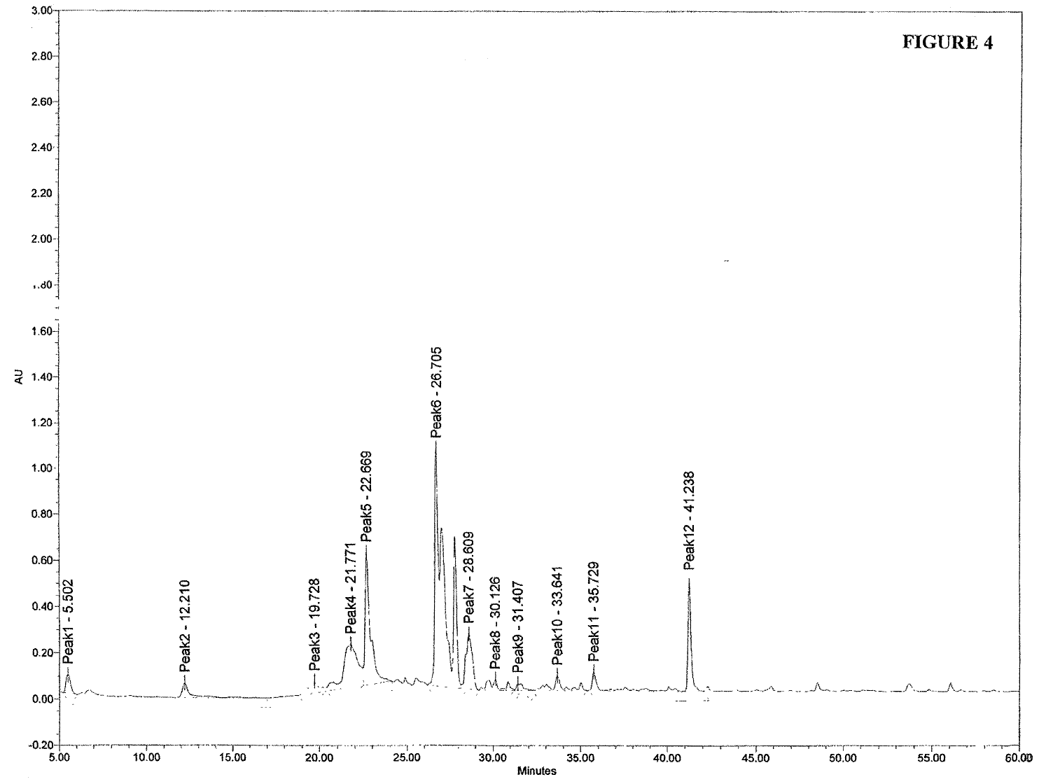

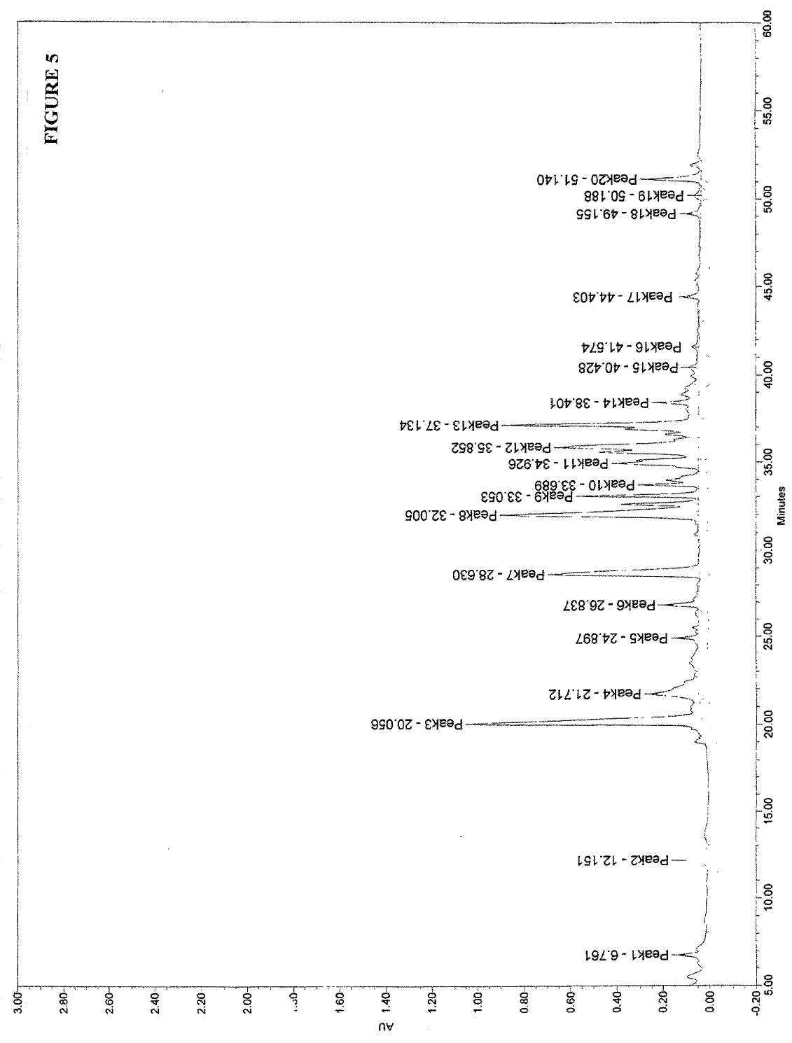

The following standard HPLC fingerprints show extracts of Arctium lappa L., FIG. 3. Carrot, FIG. 4. Radish and radish leaves, FIG. 5.

C18 column, 300A, 5 uM, 4.6 mmID×250 mmL, Flow rate 1 mL/min, solvent A: 0.1% TFA/Water, solvent B: 0.1% TFA/Acetonitrile. 0-10 min 100% solvent A, 10-70 min 0%-50% solvent B, 70-100 min 50%-100% solvent B, 100-110 min 100%-0% solvent B.

Quality Control of Raw Materials

Each tablet contains 500 mg. Administering one or two tablets, two times a day to a person in need of treatment provides an improved condition.

The mixed vegetable tablet of this invention is useful in view of pharmacological properties. In particular, it is an active anti-hypertensive agent, as evidenced by its ability to reduce blood pressure in mammals in which blood pressure has become abnormally elevated. The mixed vegetable tablet of this invention also shown activity as a detoxifier, and as anti-constipation agents.

The following examples used Formula 2.

### EXAMPLE 1

A 65-year-old female has had a history of hypertension for more than ten years. Her blood pressure was around 140 to 165. After she took two mixed vegetable tablets twice a day, her systolic blood pressure number decreased from 156 to 129 and her diastolic dropped from 88 to 73. She continued to ingest the same amount for 3 days, and her blood pressure stabilized to around 129/73.

### EXAMPLE 2

A 43-year-old male with hypertension. He had been on medication since he started having symptoms. He stopped all medication and ingested mixed vegetable tablets, two times a day. His diastolic phase blood pressure dropped from 110 to 90. He continued taking two tablets, twice a day, for ten days, and the reading stabilized at 90.

### EXAMPLE 3

A 49-year-old male had a constipation problem. After using mixed vegetable tablets, he started having normal bowel movements.

### EXAMPLE 4

A 44-year-old male ingested two mixed vegetable tablets, twice a day, and noticed increased bowel movements and urination. This is a demonstration of successful detoxification.

### CONCLUSION, RAMIFICATIONS, AND SCOPE

The mixed vegetable tablet disclosed in this invention is a unique medicine. It is simple to use and safe, without any side effects. It is a good dietary supplement for adults and seniors.

The mixed vegetable medicine can be any shape, size and color are subject to possible substitution.

What is claimed is:

1. A method of detoxification in a human comprising:
(a) Extracting arctium lappa L., carrot, whole radish with water one or two hours at temperatures of 70° C.-100° C. under agitation, separating the extracts and solid by-products, vacuum condensing the extracts, lyophilizing condensed extracts to powder using a low temperature, encapsulating the powder or pressing the powder into tablets.
(b) Having said human ingest tablets produced by the method of step (a) in a treatment program to improve health conditions and said detoxification.

2. A method of treating constipation of a patient comprising:
(c) Extracting arctium lappa L., carrot, whole radish with water one or two hours at temperatures of 70° C.-100° C. under agitation, separating the extracts and solid by-products, vacuum condensing the extracts, lyophilizing condensed extracts to a powder using a low temperature, and encapsulating the powder or pressing the powder into tablets.
(d) Having said human ingest tablets produced by the method of step (c) in a treatment program sufficient to treat said constipation of said patient.

3. A method of cure hypertension in a patient comprising:
(e) Extracting arctium lappa L., carrot and whole radish with water one or two hours at temperatures of 70° C.-100° C. under agitation, separating the extracts and solid by-products, vacuum condensing the extracts, lyophilizing condensed extracts to a powder using a low temperature, and encapsulating the powder or pressing the powder into tablets.
(f) Having said patient ingest tablets produced by the method of step (e) in a treatment program sufficient to reduce the blood pressure level of said patient.

4. A method of extracting arctium lappa L. and carrot with water one or two hours at a temperature of 100° C. and identifying the composition by High Pressure Liquid Chromatography, as shown in FIG. 1.

5. A method of extracting arctium lappa L., carrot and whole radish with water one or two hours at a temperature of 100° C. and identifying the composition of extracts by High Pressure Liquid Chromatography, shown as FIG. 2.

6. A method of extracting arctium lappa L. with water one or two hours at a temperature of 100° C. and identifying the composition of extracts by High Pressure Liquid Chromatography, shown as FIG. 3.

7. A method of extracting carrot with water one or two hours at a temperature of 100° C. and identifying the composition of extracts by High Pressure Liquid Chromatography, as shown in FIG. 4.

8. A method of extracting whole radish with water one or two hours at a temperature of 100° C. and identifying the composition of extracts by High Pressure Liquid Chromatography, as shown in FIG. 5.
